(19) United States
(12) Patent Application Publication
(10) Pub. No.: US 2009/0143630 A1
(43) Pub. Date: Jun. 4, 2009

(54) Diisobutene process

(76) Inventors: Walter S. Dubner, Wilmington, DE (US); Christopher P. Renaudo, Houston, TX (US); Shaw-Chan Lin, West Chester, PA (US)

Correspondence Address: LyondellBasell Industries, 3801 WEST CHESTER PIKE, NEWTOWN SQUARE, PA, 19073, US

(21) Appl. No.: 11/999,222

(22) Filed: Dec. 4, 2007

(51) Int. Cl. C07C 2/08

(52) U.S. Cl. 585/506

(57) ABSTRACT

A process comprising reacting a feed comprising isobutene and a modifier in the presence of an acidic solid catalyst to produce diisobutene is disclosed. The amount of the modifier in the feed is reduced as the catalyst ages. Lower reaction temperature is maintained throughout the catalyst life, which is less likely to corrode the reactor. When an alcohol is used as a modifier, a lower reaction temperature causes lower degree of dehydration of the modifier, thus lower water concentration is maintained, which in turn reduces the likelihood of reactor corrosion.

### FIELD OF THE INVENTION

The invention is a process for making diisobutene from isobutene in the presence of an acidic solid catalyst. Diisobutene may be used as fuel blending components.

### BACKGROUND OF THE INVENTION

The dimerization of olefins such as isobutene using an acidic solid catalyst is well-known in the art. For instance, U.S. Pat. No. 4,100,220 describes isobutene dimerization using a sulfonic acid resin catalyst and tert-butanol (tert-butyl alcohol, TBA) modifier. U.S. Pat. No. 4,447,668 discloses isobutene dimerization using sulfonic acid resin Amberlyst-15 with methyl tert-butyl ether as solvent. U.S. Pat. No. 5,877,372 describes the dimerization of isobutene using a sulfonic acid resin catalyst, tert-butanol modifier, and isooctane diluent. U.S. Pat. No. 6,376,731 discloses the dimerization of isobutene in the presence of a C₃ or C₄ alkane and tert-butanol to promote selectivity to diisobutene.

The diisobutene produced may be used as such or may be hydrogenated to isooctane as described in U.S. Pat. No. 5,877,372 and U.S. Pat. No. 6,376,731. Diisobutene and isooctane are useful fuel blending components.

Nearly all solid catalysts deactivate with time on stream. Often the reaction temperature needs to be raised as the catalyst deactivates so as to maintain a constant product throughput. In an isobutene dimerization catalyzed by an acidic solid catalyst, small amounts of acid may leach into the reaction media, potentially corroding the reactor.

### SUMMARY OF THE INVENTION

The invention is an isobutene dimerization process. The process comprises reacting a feed comprising isobutene and a modifier in the presence of an acidic solid catalyst, wherein the concentration of the modifier in the feed is reduced as the catalyst deactivates. Reactor throughtput is maintained without raising reaction temperature, which could promote reactor corrosion.

### BRIEF DESCRIPTION OF DRAWING

FIG. 1 shows the net TBA (wt. %) in isobutene dimerization as a function of the reaction temperature, initial TBA concentration in the feed, and isobutene conversion.

### DETAILED DESCRIPTION OF THE INVENTION

The invention is a process comprising reacting a feed comprising isobutene and a modifier in the presence of an acidic solid catalyst to produce diisobutene. Suitable solid catalysts include acidic ion-exchange resins, mixed metal oxides (e.g., silica-alumina), acidic zeolites, acidic clays, and mixtures thereof.

Preferred catalysts are acidic ion-exchange resins. Acidic ion-exchange resins generally contain sulfonic acid or carboxylic acid groups. The acidic ion-exchange resin may contain protons and other cations (e.g., alkali metal, alkaline earth metal, ammonium). Sulfonic acid resins, which are well known, are most preferred. Commercially available sulfonic acid resins include Amberlyst A-15, Amberlyst A-35, Amberlyst A-36 (available from Rohm & Haas Company), Purolite® C-275 (available from Purolite Corporation), and Dowex® 50 (available from Dow Chemical Company). Preferably the sulfonic acid resin contains from 5 to 30 weight percent (wt. %) sulfur. The dimerization of isobutene using sulfonic acid resins is known in the art and has been described in U.S. Pat. Nos. 4,100,220, 4,447,668, 5,877,372 and 6,376,731, the teachings of which are incorporated herein by reference.

The feed comprises isobutene. Isobutene in the feed may be from a number of sources. Suitable sources include isobutene-containing streams from refining or steam cracking units, such as the refinery Cat B-B and Raffinate-1, or pure isobutene from TBA dehydration as described in U.S. Pat. Nos. 5,625,109, 3,510,538, 4,165,343, and 4,155,945. The production of TBA by the Oxirane process is well known, see, for example, U.S. Pat. No. 3,351,635. Cat B-B (sometimes known as Refinery B-B) is a C₄ stream (primarily butenes and butanes) from the refining of crude oil by fluid catalytic cracking (FCC). Raffinate-1 is produced in steam cracking units after the selective separation or selective hydrogenation of 1,3-butadiene (see U.S. Pat. No. 6,586,649).

The amount of isobutene in the feed may range from approximately 5 wt. % to 99.5 wt. %. Preferably it contains at least 10 wt. % isobutene, more preferably, at least 50 wt. %.

The feed may comprise a diluent. A diluent is used as a heat sink to reduce the temperature rise from the heat of the reaction. Typically, a C₁₋₁₀ paraffin (saturated hydrocarbon) is used. Suitable diluents include propane, butanes, pentanes, hexanes, heptanes, octanes, and mixtures thereof. For example, U.S. Pat. No. 5,877,372 discloses the oligomerization of isobutene in the presence of an isoalkane diluent. U.S. Pat. No. 6,376,731 discloses an isobutene dimerization process in the presence of a C₃ or C₄ diluent. Preferably, the feed contains from 1 to 90 wt. % diluent, more preferably from 10 to 50 wt. % diluent.

The feed comprises a modifier. A modifier is a compound that moderates the catalyst activity and improves its selectivity. Generally the modifier is an oxygenate (an organic molecule containing oxygen). Suitable oxygenates include alcohols, ethers, ketones, esters, phenols, and the like. Preferably, an alcohol is used. TBA is particularly preferred. The amount of modifier is preferably at least 0.5 wt. % relative to the feed, preferably from 1 to 15 wt. %, most preferably from 3 to 10 wt. %. Water may be used as a modifier, as water can react with isobutene to form TBA under the reaction conditions.

A portion of the product stream may be recycled back to the reactor. Recycling helps to control the reaction temperature, as the recycled stream dilutes the feed and lowers the concentration of the isobutene in the reactor.

It is well known that activity of a catalyst often decreases as it is being used in a chemical process. Causes of solid catalyst deactivation are basically threefold: chemical, mechanical, and thermal. Mechanisms of solid catalyst deactivation can be classified into five general modes: (1) chemical degradation including volatilization and leaching, (2) fouling, (3) mechanical degradation, (4) poisoning, and (5) thermal degradation. See Bartholomew, C. H. and Farrauto, R. J., Fundamentals of Industrial Catalytic Processes, second edition, John Wiley & Sons (2006) pp. 260-287. In isobutene dimerizations catalyzed by acidic solid catalysts, the catalyst deactivation may be caused by leaching of acidic species from the catalyst, fouling of catalyst surface due to the formations of oligomers or polymers, attrition of the catalyst, poisoning of the acidic sites by impurities (e.g., ammonia or amines), and thermal degradation of the catalyst. It is well known, for example, that sulfonic acid resins decompose at high temperature, producing sulfonic or sulfuric acids.

In the present process, the concentration of the modifier in the feed is adjusted as the catalyst deactivates. Optionally, the reactor temperature may be adjusted as well. Usually, in a continuous process, the temperature of the reactor is raised as the catalyst deactivates so as to maintain the flow rate and the conversion constant. Other strategies may be used to deal with the catalyst deactivation in a commercial plant, as described in Chem. Eng. J. 28 (1984) 13, which include: (1) varying throughput of the reactor feed while holding the reactor temperature and conversion constant; (2) allowing the conversion to fall while holding the reactor feed flow and the reactor temperature constant; (3) maintaining the fresh feed rate and the reactor temperature constant and let the recycle flow increase; (4) using a combination of parallel reactors so that one of reactors will be off-line, so the catalyst may be regenerated or replaced with fresh catalyst while the other reactors are operating; (5) continuous catalyst regeneration while maintaining throughput and the reactor temperature. Option (1) or (2) reduces the production rate as the catalyst deactivates. Option (3) may be limited by the equipment size (e.g., recycle pump, pressure drop across the bed, etc.). Option (4) requires additional reactors, thus greater capital investment. Option (5) can only be used in certain reactor types (e.g., fluidized-bed or slurry reactors) where a portion of the catalyst may be removed relatively easily from the reactor and the regenerated catalyst or fresh catalyst may be added to the reactor without shutting down the operation. Removing catalyst from a continuously operated reactor is a troublesome operation. In a fixed-bed process, such operation is extremely difficult to implement.

If an alcohol (e.g., TBA) is used as a modifier, a portion of the modifier may be dehydrated under the reaction conditions to produce water. For example, TBA is converted to isobutene and water. Water in the reaction media, particularly when it forms a separate phase and contains free acids leached from the catalyst, may corrode the reactor. The higher the reaction temperature, the more water forms, and the higher the risk of reactor corrosion. In such a case, lower reaction temperature is particularly beneficial.

The temperature of the dimerization partly depends on the type of catalyst used. The isobutene dimerization may be conducted at a temperature in the range of from 0 to 200° C., preferably from 20 to 150° C., most preferably from 50 to 120° C., and under a pressure sufficient to maintain the reactor content in liquid phase, preferably above 50 psig, e.g., from 50 to 500 psig.

The process may be performed in a batch, semi-batch, or a continuous mode. Preferably, the process is conducted in a continuous mode where the reactants continuously flow in the reactor and the products continuously flow out of the reactor (Smith, J. M., Chemical Engineering Kinetics, third edition, McGraw-Hill, Inc. (1981) pp. 25-33). The catalyst may be in a fixed bed or a slurry. A continuous fixed-bed process is particularly preferred.

The reaction products include diisobutene as well as some non-reacted isobutene and isobutene oligomers (e.g., triisobutenes, tetraisobutenes). Diisobutene and isobutene may be separated with conventional techniques (e.g., distillation). The isolated isobutene from the product stream may be recycled back to the dimerization.

The following examples illustrate the invention.

### EXAMPLE 1-A

A 500-mL autoclave reactor is equipped with a feed line, a product line, a thermo well, and a stirrer. Purolite® CT 275 (Purolite Corporation, 20 g) is charged to the reactor. A feed consisting of 4.03 wt. % TBA and 95.97 wt. % isobutylene is continuously fed to the reactor. The product stream exits the reactor from the product line. The weight hourly space velocity is controlled at 2 h⁻¹. The reactor is heated with an electric heater and the temperature of the reaction is controlled at 150° F. The product stream is analyzed by an on-line gas chromatography (GC). The isobutylene conversion is 59%. The TBA concentration in the product stream is 4.33 wt. %. The results are listed in Table 1. The net TBA made by the reaction is 0.30 wt. %.

### EXAMPLES 1-B TO 1-E

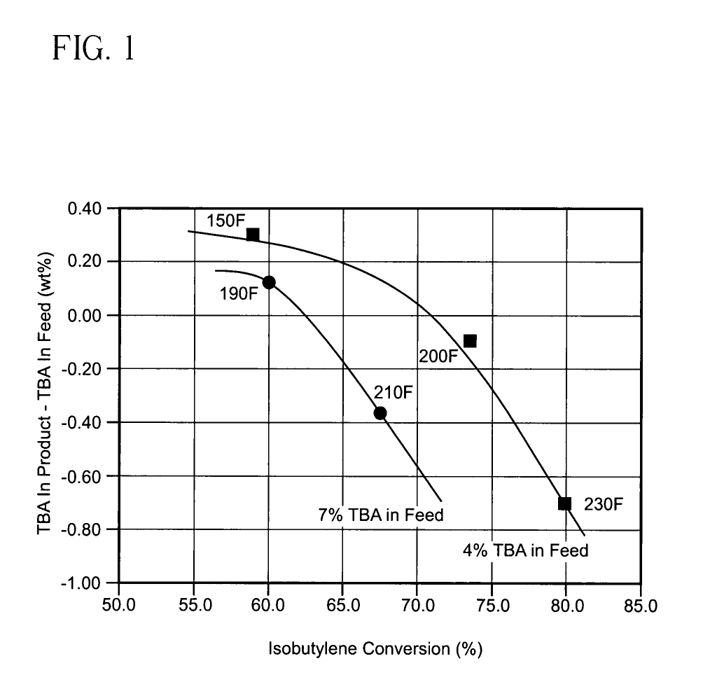

The procedure of Example 1 is repeated, except that the amounts of TBA and isobutylene fed to the reactor and the reaction temperature are different. The detailed reaction conditions and the results are shown in Table 1, and graphed in FIG. 1, where ΔTBA=TBA wt. % in product−TBA wt. % in the feed. A positive value for ΔTBA indicates that TBA is made by the process from the reaction between isobutene and water present in the feed. A small amount of water is present in isobutene and TBA. A negative value for ΔTBA indicates that TBA is dehydrated to form isobutene and water in a 1:1 molar ratio.

FIG. 1 shows that at constant TBA concentration in the feed, the higher the reaction temperature, the more TBA is converted to isobutene and water. In addition, higher temperature can cause the thermal decomposition of the catalyst. Thus in general, lower reaction temperature is preferred. FIG. 1 also shows that at the same isobutene conversion, the lower the TBA concentration in the feed, the lower the reaction temperature required. According to the present invention, as the catalyst deactivates, the TBA is lowered so as to maintain the constant isobutene conversion.

### EXAMPLE 2

A mixture of isobutene (flow rate=100 g/h) and TBA (flow rate=7 g/h) is fed to the top of a 0.8″ ID tube reactor containing Purolite® CT-275 (washed with methanol and dried under vacuum at 120° C., 50 g). The reactor is under a pressure of 300 psig. The product exits the reactor from the bottom of the reactor. The temperature of the bed is slowly raised over a period of 48 h to 170° F. to control the isobutene conversion to be about 60%. The conversion is maintained constant over 3600 h. The TBA flow rate is reduced as the catalyst deactivates over time. The expected diisobutene selectivity is about 94%. Diisobutene selectivity is defined as 2×(moles of diisobutene formed)/(moles of isobutene reacted).

TABLE 1

| | | | Isobutylene | | |
|---|---|---|---|---|---|
| | Feed TBA | T | Conversion | Product TBA | ΔTBA |
| Example | (wt. %) | (° F.) | (%) | (wt. %) | (wt. %) |
| 1-A | 4.03 | 150 | 59.0 | 4.33 | 0.30 |
| 1-B | 4.03 | 200 | 73.5 | 3.93 | −0.10 |
| 1-C | 4.03 | 230 | 79.9 | 3.22 | −0.81 |
| 1-D | 7.22 | 190 | 60.1 | 7.34 | 0.12 |
| 1-E | 7.06 | 210 | 67.6 | 6.69 | −0.37 |

We claim:

1. A process comprising reacting a feed comprising isobutene and a modifier in the presence of an acidic solid catalyst to produce diisobutene, wherein the concentration of the modifier is reduced with time as the catalyst deactivates.

2. The process of claim 1 wherein the feed contains at least 10 wt. % isobutene.

3. The process of claim 1 wherein the feed contains at least 50 wt. % isobutene.

4. The process of claim 1 wherein the feed contains a hydrocarbon diluent.

5. The process of claim 1 wherein the catalyst is an ion-exchange resin.

6. The process of claim 1 wherein the catalyst is a sulfonic acid ion-exchange resin.

7. The process of claim 6 wherein the catalyst contains from 5 to 30 wt. % sulfur.

8. The process of claim 1 wherein the modifier is an oxygenate.

9. The process of claim 1 wherein the modifier is an alcohol.

10. The process of claim 1 wherein the modifier is tert-butanol (TBA).

11. The process of claim 10 wherein the amount of TBA is from 1 to 15 wt. % relative to the amount of the feed.

12. The process of claim 10 wherein the amount of TBA is from 3 to 10 wt. % relative to the amount of the feed.

13. The process of claim 1 performed at a temperature in the range of 50 to 120° C.

14. A fixed-bed process of claim 1.

15. A continuous process of claim 1.
